SEQUENCE LISTING (10 sequences)

SEQ ID NO: 1 - protein, 248 aa, Artificial Sequence
  antibody with homology to fungal stress protein hsp 90
HMAEVQLVESGAEVKKPGESLRISCKGSGCIISSYWISWVRQMPGKGLEWMGKIDPGDSYINYSPSFQGHVTISADKSINTAYLQWNSLKASDTAMYYCARGGRDFGDSFDYWGQGTLVTVSSGGGGSGGGGSGGGGSDVVMTQSPSFLSAFVGDRITITCRASSGISRYLAWYQQAPGKAPKLLIYAASTLQTGVPSRFSGSGSGTEFTLTINSLQPEDFATYYCQHLNSYPLTFGGGTKVDIKRAA

SEQ ID NO: 2 - protein, 707 aa, Candida albicans
MADAKVETHEFTAEISQLMSLIINTVYSNKEIFLRELISNASDALDKIRYQALSDPSQLESEPELFIRIIPQKDQKVLEIRDSGIGMTKADLVNNLGTIAKSGTKSFMEALSAGADVSMIGQFGVGFYSLFLVADHVQVISKHNDDEQYVWESNAGGKFTVTLDETNERLGRGTMLRLFLKEDQLEYLEEKRIKEVVKKHSEFVAYPIQLVVTKEVEKEVPETEEEDKAAEEDDKKPKLEEVKDEEDEKKEKKTKTVKEEVTETEELNKTKPLWTRNPSDITQDEYNAFYKSISNDWEDPLAVKHFSVEGQLEFRAILFVPKRAPFDAFESKKKKNNIKLYVRRVFITDDAEELIPEWLSFIKGVVDSEDLPLNLSREMLQQNKILKVIRKNIVKKMIETFNEISEDQEQFNQFYTAFSKNIKLGIHEDAQNRQSLAKLLRFYSTKSSEEMTSLSDYVTRMPEHQKNIYYITGESIKAVEKSPFLDALKAKNFEVLFMVDPIDEYAMTQLKEFEDKKLVDITKDFELEESDEEKAAREKEIKEYEPLTKALKDILGDQVEKVVVSYKLVDAPAAIRTGQFGWSANMERIMKAQALRDTTMSSYMSSKKTFEISPSSPIIKELKKKVETDGAEDKTVKDLTTLLFDTALLTSGFTLDEPSNFAHRINRLIALGLNIDDDSEETAVEPEATTTASTDEPAGESAMEEVD

SEQ ID NO: 3 - protein, 732 aa, Homo sapiens
MPEETQTQDQPMEEEEVETFAFQAEIAQLMSLIINTFYSNKEIFLRELISNSSDALDKIRYETLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 4 - protein, 6 aa, Candida albicans
LKVIRK

SEQ ID NO: 5 - protein, 9 aa, Candida albicans
LKVIRKNIV

SEQ ID NO: 6 - protein, 12 aa, Artificial Sequence
  prokaryotic or eukaryotic
  misc_feature (1)..(3): Xaa can be any naturally occurring amino acid
  misc_feature (5)..(5): Xaa can be any naturally occurring amino acid
  misc_feature (10)..(10): Xaa can be any naturally occurring amino acid
XXXLXVIRKXIV

SEQ ID NO: 7 - protein, 12 aa, Artificial Sequence
  prokaryotic or eukaryotic
  misc_feature (1)..(2): Xaa can be any naturally occurring amino acid
  misc_feature (5)..(5): Xaa can be any naturally occurring amino acid
  misc_feature (8)..(12): Xaa can be any naturally occurring amino acid
XXILXVIXXXXX

SEQ ID NO: 8 - DNA, 19 bp, Candida albicans
atggctgacgcaaaagttg

SEQ ID NO: 9 - DNA, 20 bp, Candida albicans
atcaacttcttccatagcag

SEQ ID NO: 10 - protein, 6 aa, Candida albicans
DEPAGE

(19) United States
(12) Patent Application Publication
(10) Pub. No.: US 2007/0154478 A1
(43) Pub. Date: Jul. 5, 2007

(54) Therapeutic composition

(75) Inventors: James Peter Burnie, Alderley Edge (GB); Ruth Christine Matthews, Alderley Edge (GB)

Correspondence Address: ROTHWELL, FIGG, ERNST & MANBECK, P.C., 1425 K STREET, N.W., SUITE 800, WASHINGTON, DC, 20005, US

(73) Assignee: NeuTec Pharma PLC, Manchester (GB)

(21) Appl. No.: 11/401,321

(22) Filed: Apr. 11, 2006

(30) Foreign Application Priority Data
Jan. 5, 2006 (GB) 0600168.9

(51) Int. Cl. A61K 39/395; G01N 33/53

(52) U.S. Cl. 424/145.1; 435/7.1

(57) ABSTRACT

Use of an inhibitor of an hsp 90 protein for the manufacture of a medicament for the treatment or prophylaxis of a condition involving raised levels of TNFα and/or IL-6.

### TECHNICAL FIELD

The present invention relates to a medicament for a therapeutic treatment or prophylaxis of a condition involving raised levels of TNFα and/or IL-6. The invention also relates to a method of lowering TNFα and/or IL-6 levels in a patient; and also to a method of diagnosing conditions involving raised levels of TNFα and/or IL-6.

### BACKGROUND ART

Sepsis is a serious medical condition, typically caused by a severe infection which can lead to a systemic inflammatory response. Symptoms may include fever, chills, malaise, and low blood pressure. Even when receiving treatment, a patient suffering from sepsis may progress to multiple organ dysfunction syndrome or even death.

The symptoms of sepsis are also observed to arise in circumstances where infection is known not to have occurred and in such cases the condition is known as Systemic Inflammatory Response Syndrome (SIRS).

Interleukin 6 (IL-6) is part of the acute-phase response in infection such that a raised level in a patient has been correlated with more severe infection and a poorer outcome for the patient. Recently, raised IL-6 levels have been reported as being associated with sepsis and SIRS.

In neonates at an optimal cut off of 31 pg/ml a raised IL-6 level had a sensitivity of 89% and negative predictive value of 91% for detecting late onset infection on day 0 (Ng et al 1997). Levels of IL-6 were significantly higher in fungal infections when compared with Gram positive sepsis (p=0.035) and there was a very elevated level in an infant who died from fungal sepsis (Ng et al 2003). In surgical patients a raised IL-6 was associated with SIRS (Miyaoka et al 2005) and at a cut off of 310 pg/ml in patients with septic complications in their first five postoperative days yielded the test had a sensitivity of 90% and specificity of 58% when differentiating between patients with and without post operative septic complications (Mokart et al 2005). A raised IL-6 was associated in patients with SIRS and presumed infection (mean 222.8 pg/ml) as compared with SIRS presumed non infectious (mean 80.9 pg/ml) (Terregino et al 2000).

Interleukin 6 production is induced in part by tumour necrosis factor (TNF-α). It has been reported in the art to neutralize TNF-α for therapeutic purposes and to use levels of interleukin 6 as a surrogate marker of TNF-α activity. For example, in a study of the efficacy and safety of a monoclonal anti-TNF-a antibody F (ab′)2 (known as Afelimomab) activity was apparent in patients with a high interleukin 6 level and absent in patients who were interleukin 6 negative (Panacek et al 2004). Such proposed therapies are based on the theory that TNF-α is the host damaging cytokine and that LPS (lipopolysaccharide) triggers TNF-α release and this leads to septic shock developing (Hehlgans and Pfeffer 2005). This theory is based on the observation that high levels of TNF-α are present during sepsis, where they predict death of a patient, whilst falling levels of TNF-α correlate with survival of the patient.

A separate area of study has been the development of the drug Mycograb® which comprises an antibody against the fungal stress protein hsp 90. This was developed following the observation that patients with invasive candidiasis sero-convert to hsp 90 when they recover from the disease. WO-A-01/76627 reports on the use of a combination of the Mycograb® antibody and a polyene (such as amphotericin B) or an echinocandin antifungal agent in order to treat fungal infections. It has also been reported that a combination of the drug and amphotericin B showed a synergistic effect, when compared with amphotericin B and placebo (saline) in clinical trials, due to its direct activity as an anti-fungal and the ability of the drug to neutralise circulating hsp 90. Matthews et al. 2005 reported on what role hsp 90 might play in human disease.

The present invention is based on the finding that administering hsp 90 protein results in raised levels of TNFα and IL-6 and that this effect can be neutralised by prior cross absorption of hsp90 with the Mycograb® drug (but not with Aurograb® which comprises an antibody against the ABC transporter of MRSA).

While not wishing to be bound by any theory, it is believed that the invention works because the presence of hsp 90 protein circulating in an individual causes levels of TNFα and IL-6 in the individual to rise. The presence of hsp 90 protein in the individual may act directly to raise IL-6 levels in the individual or it may be that raised levels of TNFα cause levels of IL-6 in the individual to rise. The presence of higher levels of these two cytokines (TNFα and IL-6) in the individual causes the inflammatory response that is observed as sepsis or SIRS. The reasoning for this theory will now be explained.

It has been reported in the prior art that the Mycograb® drug works in treating fungal infections by neutralising the fungal hsp 90 protein. The epitope, to which the Mycograb® antibody is specific, is conserved with human hsp 90 so the Mycograb® antibody will inevitably also bind and neutralise the human hsp 90 protein. This binding has been confirmed by the data reported in Example 1 herein (Binding of Mycograb to human and fungal hsp 90).

Hsp 90 is considered to be an intra-cellular protein released only on cell necrosis and not on cell apoptosis (Saito et al 2005). It is thus proposed that necrosing cells release hsp 90 into circulation in a patient which leads to the patient presenting symptoms resembling sepsis (i.e. the SIRS-Systemic Inflammatory Response Syndrome) in the absence of a positive culture for a micro-organism. This situation is worsened in fungal sepsis where fungal hsp 90 acts as a direct mimic of human hsp 90. The situation may also be worsened in bacterial sepsis where the bacterial homologue htpG may be released and produce or worsen the clinical picture.

In sepsis the free hsp90/htpG may induce the septic picture and this can be seen indirectly by the induction of high levels of interleukin 6 as are now reported (see Example 3). Levels of interleukin 6 were measured in the sera of patients in a double blind placebo-controlled study. A reduction in IL-6 levels was correlated with recovery in the group treated with Mycograb® but this did not happen in the Placebo group. Most significantly, patients with Candida-attributable mortality in the Placebo group had persistent, high levels of IL-6.

The data reported herein supports the concept that hsp90 leads to interleukin 6 release directly so that neutralization of hsp 90 efficiently blocks IL-6 release. It also supports the concept that neutralising hsp90 blockage will block TNF-α release so that inhibiting the hsp 90 protein would be effective in the treatment of auto-immune diseases where TNF-α is the most important molecule.

According to one aspect of the present invention, there is provided the use of an inhibitor of an hsp 90 protein for the manufacture of a medicament for the treatment or prophylaxis of a condition involving raised levels of TNFα and/or IL-6.

In another aspect of the present invention, there is provided a method of lowering TNFα and/or IL-6 levels in a patient comprising administering to the patient an inhibitor of an hsp 90 protein.

In some embodiments, the patient is suffering from a condition due to raised TNFα and/or IL-6 levels.

According to a further aspect of the present invention, there is provided a method of diagnosing a condition in a patient involving raised levels of TNFα and/or IL-6 comprising the step of determining the level of an hsp 90 protein circulating in the patient, wherein a raised level of the hsp 90 protein is indicative of the presence of the condition.

Determining the level of the hsp 90 protein that is circulating in the patient may be carried out directly on the patient but is more conveniently effected by determining the levels of hsp 90 protein in a sample (eg a blood sample) taken from the patient. In this way, the diagnostic method is carried out ex vivo.

The patient is typically a mammal and most preferably a human.

A condition involving raised levels of TNFα (Tumour Necrosis Factor α) or IL-6 (i.e. interleukin-6) is one in which TNFα or IL-6, respectively, acts as a marker for the condition due to it being at above normal levels in patients suffering the condition. Further explanation of conditions involving raised levels of IL-6 and the use of IL-6 as a marker is provided in Miyaoka et al. 2005, Mokart et al. 2005, Ng 1997, Ng et al. 2003, Ng et al. 2004 and Terregino et al. 2000. Examples of such conditions include sepsis and SIRS (Systemic Inflammatory Response Syndrome). Raised levels of TNFα are involved, for example, in autoimmune diseases such as Crohn's disease, rheumatoid arthritis, ulcerative colitis and systemic lupus erythematosus (SLE).

Levels of TNFα or IL-6 in a patient can be assessed by, for instance, using the TNFα assay and the Interleukin 6 assay reported in Example 2. In some embodiments, a level of TNFα or IL-6 that is indicative of abnormal levels thereof is 5, 10 or 20 times normal concentrations in the patient. However, it is to be noted that levels several hundred times normal (eg 100 times) are observed in some patients.

It is to be appreciated that sepsis may be due to an infection or due to other causes (i.e. SIRS) and the present invention covers both instances. In some embodiments, the sepsis is as a result of fungal or bacterial infection but it is to be understood that the invention also relates to sepsis which is not due to a fungal or a bacterial infection.

Hsp 90 proteins are a family of highly conserved stress proteins which are produced in a wide range of organisms. For example, EP-A-0406029 reports on the hsp 90 protein of Candida albicans. WO-A-92/01717 reports on the hsp 90 protein of Corynebacterium jeikeium. The hsp 90 protein of homo sapiens is also known in the art and is included herein as SEQ ID NO: 3. The term “hsp 90 protein” used herein thus includes each of these proteins and also includes, for example, the bacterial homologue htpG of Escherichia coli. Furthermore, WO-A-94/04676 reports on a number of conserved sequences which are present in the hsp 90 protein of different organisms. Consequently, the present invention relates to any hsp 90 protein which falls within this family of stress proteins. In certain embodiments, the hsp 90 protein is defined more specifically as will now be explained.

In one embodiment, the hsp 90 protein comprises the amino acid sequence XXXLXVIRKXIV, wherein X is any amino acid.

In an alternative embodiment, the hsp 90 protein comprises the amino acid sequence XXILXVIXXXXX, wherein X is any amino acid.

It is to be appreciated that the above two consensus sequences are reported in WO-A-94/04676 and it is to be understood that in other embodiments of the present invention, the hsp 90 protein is defined by any of the other consensus sequences reported in WO-A-94/04676, which is hereby incorporated by reference.

In some other embodiments, the hsp 90 protein comprises the amino acid sequence LKVIRK, preferably LKVIRKNIV.

In some further embodiments, the hsp 90 protein has at least 50%, 60%, 70%, 80%, 90% or 95% identity to the sequence of hsp 90 from Candida albicans, i.e. SEQ ID NO: 2.

In this regard, it is to be appreciated that the sequence of the hsp 90 protein of Candida albicans has 58% identity with the sequence of the human hsp 90 alpha isoform 2 and consequently, a level of at least 58% identity to the sequence of the hsp 90 protein of Candida albicans is also a definition of hsp 90 proteins according to the invention.

In this specification, the percentage “identity” between two sequences is determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL www.ncbi.nlm.nih.gov/blast.

The inhibitor of the hsp 90 protein may be any protein, peptide, nucleic acid, oligonucleotide, oligosaccharide or other biologically-compatible product which is capable of lowering the activity of the hsp 90 protein in vivo. More specifically, the inhibitor lowers the action of the hsp 90 protein in raising IL-6 levels. Thus the effectiveness of a biologically-compatible product as an inhibitor of an hsp 90 protein can be assessed by determining levels of circulating hsp 90 protein in a patient with and without the product or by determining circulating levels of IL-6 in a patient with and without the product.

In some embodiments, the inhibitor comprises an antibody or an antigen-binding fragment thereof. However, this is not essential to the invention and the inhibitor may be another type of active ingredient such as the antibiotics geldanamycin, radicicol or novobiocin or the drug cisplatin.

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999.

The antibodies may be generated using standard methods known in the art. Examples of antibodies include (but are not limited to) polyclonal, monoclonal, chimeric, single chain, Fab fragments, fragments produced by a Fab expression library, and antigen binding fragments of antibodies.

An “antigen-binding fragment” includes any fragment of an antibody which is capable of binding a target antigen and thus includes Fab fragments and F(ab′)₂ fragment.

Antibodies may be produced in a range of hosts, for example goats, rabbits, rats, mice, humans, and others. They may be immunized by injection with heat shock protein from the Candida genus, for example hsp90 from C. albicans, or any fragment or oligopeptide thereof which has immunogenic properties. As another example, the host may be immunised with heat shock protein from homo sapiens. Depending on the host species, various adjuvants may be used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful.

Monoclonal antibodies to the hsp 90 heat shock protein or any fragment or oligopeptide thereof may be prepared using any technique which provides for the production of antibody molecules by continuous cell lines in culture. These include, but are not limited to, the hybridoma technique, the human B-cell hybridoma technique, and the EBV-hybridoma technique (Koehler et al., 1975, Nature, 256: 495-497; Kosbor et al., 1983, Immunol. Today 4: 72; Cote et al., 1983, PNAS USA, 80: 2026-2030; Cole et al., 1985, Monoclonal Antibodies and Cancer Therapy, Alan R. Liss Inc., New York, pp. 77-96).

In addition, techniques developed for the production of “chimeric antibodies”, the splicing of mouse antibody genes to human antibody genes to obtain a molecule with appropriate antigen specificity and biological activity can be used (Morrison et al., 1984, PNAS USA, 81: 6851-6855; Neuberger et al., 1984, Nature, 312: 604-608; Takeda et al., 1985, Nature, 314: 452-454). Alternatively, techniques described for the production of single chain antibodies may be adapted, using methods known in the art, to produce hsp 90 heat shock protein-specific single chain antibodies. Antibodies with related specificity, but of distinct idiotypic composition, may be generated by chain shuffling from random combinatorial immunoglobin libraries (Burton, D. R., 1991, PNAS USA, 88: 11120-11123).

Antibodies may also be produced by inducing in vivo production in the lymphocyte population or by screening recombinant immunoglobulin libraries or panels of highly specific binding reagents (Orlandi et al., 1989, PNAS USA, 86: 3833-3837; Winter, G. et al., 1991, Nature, 349: 293-299).

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., 1989, Science, 256: 1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between an hsp 90 heat shock protein, or any fragment or oligopeptide thereof and its specific antibody. A two-site, monoclonal-based immunoassay utilizing monoclonal antibodies specific to two non-interfering hsp 90 heat shock protein epitopes may be used, but a competitive binding assay may also be employed (Maddox et al., 1983, J. Exp. Med., 158: 1211-1216).

Advantageously, the antibody or antigen-binding fragment is capable of binding or being specific for an epitope having the amino acid sequence LKVIRK, preferably LKVIRKNIV.

In some embodiments, the antibody comprises the sequence of the antibody component of Mycograb® i.e. SEQ ID NO: 1.

In order to determine the level of the hsp 90 protein in the diagnostic method, an antibody that is capable of binding the hsp 90 protein (or an antigen binding fragment thereof) is used in some embodiments. The antibody or antigen binding fragment is, for example, bound to a fluorescent tag to permit visualisation of the binding to the hsp 90 protein and thus the level (concentration or absolute amount) of the hsp 90 protein that is present. The antibodies described above in relation to the hsp 90 protein inhibitor may thus also be used in the diagnostic method.

In some further embodiments of the diagnostic method, in which the condition to be diagnosed is a pathogenic infection, the species responsible for the infection is also determined. One way by which this may be effected is by determining the sequence of the species-specific epitope which exists at the carboxy-end of the hsp 90 protein. For example, the fungal species Candida albicans has the peptide sequence DEPAGE at the species-specific epitope (see amino acid residues 695 to 700 of SEQ ID NO: 2) and thus the binding of an antibody specific for this epitope is indicative of the presence of Candida albicans as the infectious pathogen.

However, it is to be noted that the diagnostic method is not limited to diagnosing conditions which result from infection by a pathogen. Indeed the diagnostic method is particularly useful in conditions such as SIRS which arise without a pathogen being present.

Methods which can be used to manufacture the medicaments of the invention are well known. For example, a medicament may comprise, in addition to the inhibitor of an hsp 90 protein, a pharmaceutically acceptable carrier, diluent or excipient (Remington's Pharmaceutical Sciences and US Pharmacopoeia, 1984, Mack Publishing Company, Easton, Pa., USA). The exact dose (i.e. a pharmaceutically acceptable dose) of the medicament to be administered to a patient may be readily determined by one skilled in the art, for example by the use of simple dose-response experiments. The medicament may be administered orally.

### BRIEF DESCRIPTION OF THE DRAWINGS

In this specification, reference will be made to the following drawings.

FIG. 1 shows a graph of binding curves from the injection of different concentrations of Mycograb® over immobilised peptide.

FIG. 2 shows a graph of binding curves from injection of different concentrations of Mycograb® over Candida hsp90.

FIG. 3 shows a graph of binding curves from the injection of a concentration series of Mycograb® over immobilised human hsp90α.

FIG. 4 shows a graph of sensor grams showing the binding of Mycograb® to the LKVIRK-peptide at different temperatures.

FIG. 5 shows an image of a gel analysis of IMAC purification of recombinant hsp90. The lanes of the gel are as follows: Lane 1—Flow through; Lane 2—Wash 1a; Lane 3—Wash 1b; Lane 4—Wash 1c; Lane 5—Wash 1d; Lane 6—Wash 1e; Lane 7—Elution 1a; and Lane 8—Elution 1b.

FIG. 6 shows a graph of mouse response to hsp 90 without cross-absorption by Mycograb®.

FIG. 7 shows a graph of mouse response to hsp 90 with cross-absorption by Mycograb® at a concentration of 0.1 mg/kg.

FIG. 8 shows a graph of mouse response to hsp 90 with cross-absorption by Mycograb® at a concentration of 0.5 mg/kg.

FIG. 9 shows a graph of mouse response to hsp 90 with cross-absorption by Mycograb® at a concentration of 1 mg/kg.

### BRIEF DESCRIPTION OF THE SEQUENCE LISTINGS

SEQ ID NO: 1 is the amino acid sequence of the antibody component of Mycograb®.

SEQ ID NO: 2 is the amino acid sequence of the hsp 90 stress protein from Candida albicans.

SEQ ID NO: 3 is the amino acid sequence of the human hsp 90 alpha isoform 2 protein.

SEQ ID NO: 4 is the amino acid sequence of the epitope in hsp 90 to which the antibody of SEQ ID NO: 1 is specific.

SEQ ID NO: 5 is the amino acid sequence of the epitope of SEQ ID NO: 4 with adjacent amino acid residues.

SEQ ID NO: 6 is a consensus sequence for an epitope on hsp 90.

SEQ ID NO: 7 is a consensus sequence for an epitope on hsp 90.

SEQ ID NO: 8 is a PCR primer sequence used in the examples.

SEQ ID NO: 9 is a PCR primer sequence used in the examples.

### EXPERIMENTAL

### Example 1

### Demonstration of Mycograb Binding to the Target Epitope LKVIRK, Human and Fungal hsp90

The binding of Mycograb® to the LKVIRK-peptide (SEQ ID NO: 4) from within hsp90 against which it was originally matched, and recombinant versions of hsp90 derived from the sequences representing the homologues from Candida albicans and human hsp90α was demonstrated using real time Biacore analysis. The effect of temperature on the binding to the LKVIRK-peptide was additionally investigated.

Material and Methods

The immobilization of biotinylated LKVIRK peptide to a Sensor Chip SA was by non-covalent capture performed by running HBS-EP buffer consisting of 10 mM Hepes, 150 mM NaCl, 0.005% Tween 20 and 3.4 mM EDTA, pH 7.4 continuously over 2 adjacent flow cells at a flow rate of 20 μl/min. Candida albicans hsp90, dialysed into 10 mM sodium acetate pH 4.0, was covalently bound to the surface of a CM-5 Chip using amino coupling. Human hsp90α (1.15 mg/ml) was diluted 1:50 in 10 mM sodium acetate pH 4.0 and covalently bound to the surface of a CM-5 Chip using amino coupling.

Mycograb® was formulated as is described in WO-A-01/76627 (see, in particular, pages 11 and 12) which is hereby incorporated by reference.

Results

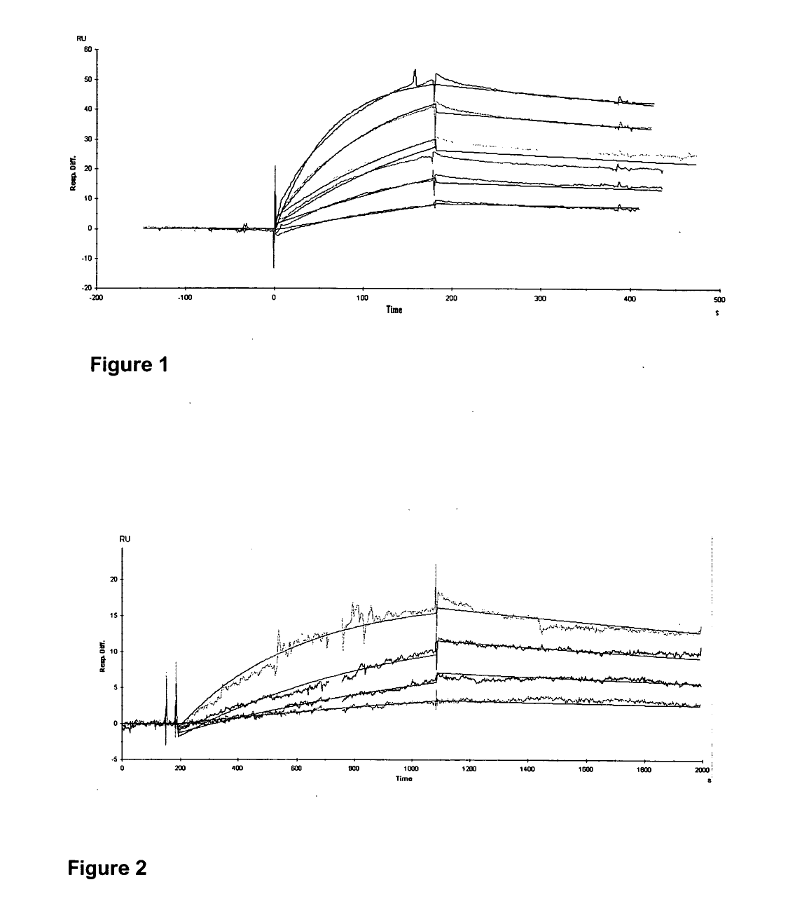

The results from analysing the binding of a concentration series of Mycograb® to the peptide are shown in FIG. 1 were evaluated using the Biacore evaluation software. A Langmuir model of 1:1 binding between ligand and analyte gave a good fit of the binding curves and kₐ (association rate constant) was calculated to be 2.26×10⁴ M⁻¹ s⁻¹ and k_{d} (dissociation rate constant) was 6.47×10⁻⁴ s⁻¹. The K_{D} (dissociation constant) was 2.86×10⁻⁸ M, which meant that the binding had a long half-life of days.

The observed rate constant (K_{obs}) was plotted against the concentration of Mycograb® to test for the presence of aggregates or solubility problems within the Mycograb® sample. This resulted in a straight line demonstrating that aggregation was not an issue.

In the case of binding to Candida hsp90, a Langmuir model of 1:1 gave a good fit of the binding curves and kₐ (association rate constant) was calculated to be 373 M⁻¹ s⁻¹ and k_{d} (dissociation rate constant) was 2.67×10⁻⁴ s⁻¹. The results are shown in FIG. 2. The K_{D} (affinity constant, ratio of kₐ and k_{d}) was 7.17×10⁻⁷ M. Chi value of the fit was 1.58 (less that 2 was a good fit).

To rule out the presence of aggregates or solubility problems with the Mycograb® sample at the concentration range used for the experiment, K_{obs} was plotted against the concentration which resulted in a straight line showing that the samples that there was no evidence of aggregation.

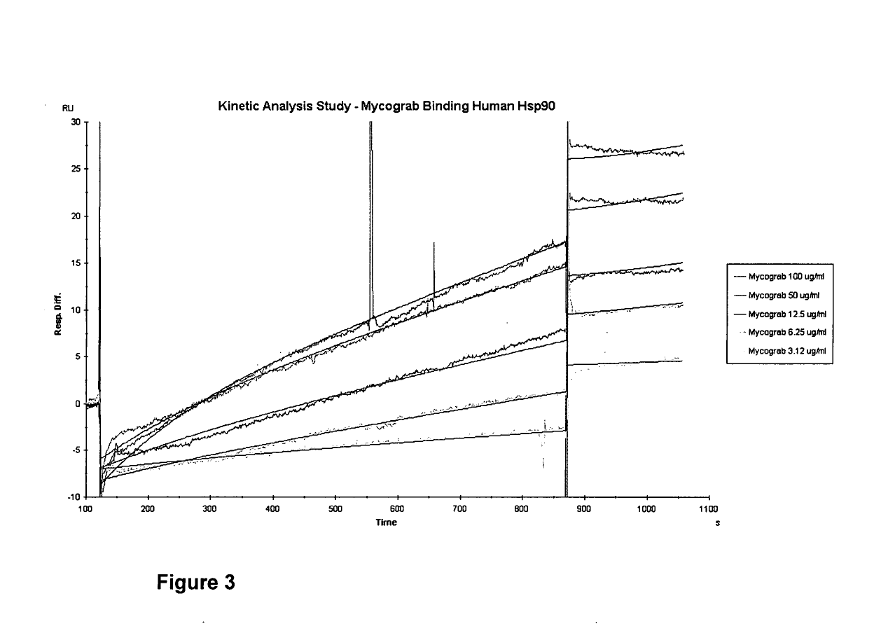

In the case of binding to human hsp90, the results of which are shown in FIG. 3, a Langmuir model of 1:1 gave a good fit of the binding curves. The model calculated kₐ as 981 M⁻¹ s⁻¹ with k_{d} 3.21×10⁻³ s⁻¹. The K_{D} was calculated as 3.27×10⁻⁶ M and the Chi value of the fit was 0.82

To rule out the presence of aggregates or solubility problems in the Mycograb® sample, k_{obs} was plotted against concentration. This resulted in a straight line demonstrating that there was no evidence of aggregation.

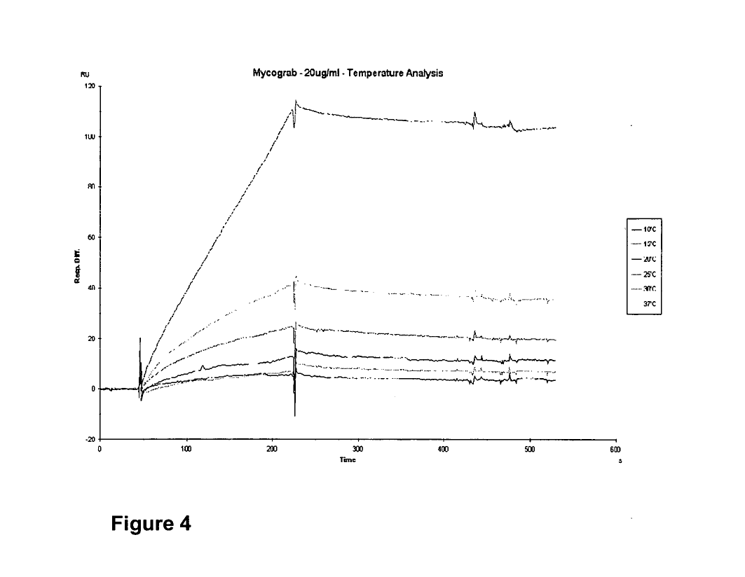

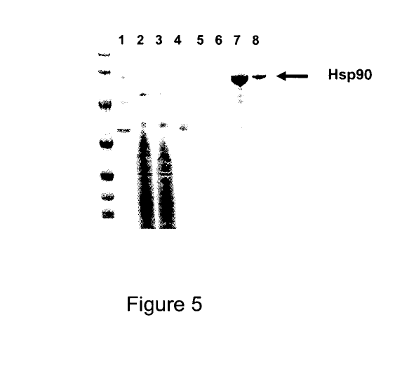

There was a clear change in the binding profile of Mycograb® to the peptide with a change in temperature in the system, the results of which are shown in FIG. 4. FIG. 4 demonstrates, in the overlaid reference subtracted sensor grams, that there was an increase in the maximum RU value with an increasing temperature. If the binding was extrapolated to the saturation point more Mycograb® bound to the surface of the chip at higher temperatures. At the low end of the temperature series, 10 to 25° C., there is a small increase in Rmax ranging from 5 to 20 RU. At 37° C. there was a significant increase in response, with the Rmax increasing to 118 RU. Inspection of the dissociation curves showed that the dissociation rate constant stayed comparatively constant irrespective of temperature.

Mycograb® bound tightly to the peptide representing the LKVIRK peptide. The association rate constant (kₐ) was 2.26×10⁻⁴ M⁻¹ s⁻¹ and when it bound it interacted strongly with its target as the dissociation rate constant (k_{d}) of 6.47×10⁻⁴ s⁻¹ shows. The K_{D} obtained was 2.86×10⁻⁸ M, which meant that the binding had a long half-life of days.

The K_{D} for the binding of Mycograb® to hsp90 from Candida albicans and human hsp90 were 7.17×10⁻⁷ M and 3.27×10⁻⁶ M respectively. Mycograb® demonstrated a clear overall lower rate of association for the hsp90 protein compared to the isolated peptide with kₐ 373 M⁻¹ s⁻¹ (Candida hsp90) and 981 M⁻¹ s⁻¹ (human hsp90) compared to 2.26×10⁴ M⁻¹ s⁻¹ (peptide). However, the k_{d} for the native interacting systems of 2.67×10⁻⁴ s⁻¹ (Candida Hsp90) and 3.21×10⁻³ s⁻¹ (human hsp90) both evoke a similarly strong interaction (long half life) on binding as for the LKVIRK peptide (6.47×10⁻⁴ s⁻¹).

Native hsp90 is a considerably larger macromolecule of approximately 80 kDa which will reduce the probability of Mycograb® reaching the specific binding site within a specified time frame, and hence reducing the kₐ. The macromolecular structure of hsp90 will significantly modify the electrostatic environment of the epitope in comparison to the isolated peptide alone. However, once successfully docked Mycograb® will sufficiently maintain the interaction irrespective of the epitope context generating similar k_{d} characteristics for the three different test systems.

There was an increase in the binding of Mycograb® to peptide at higher temperatures. The best binding was observed at 37° C. which was the temperature that Mycograb® was used at in patients. Since Mycograb® was based on a structure optimized by the human immune system it would be predicted that the binding was most efficient at body temperature.

### Example 2

### Induction of Interleukin 6 in a Murine Model

This experiment was designed to measure the production of TNF-α and Interleukin 6 in mice following the injection of purified Candida hsp90. The ability to neutralise this phenomenon by cross absorbing with Mycograb at 37° C. for 15 minutes prior to injection was also tested.

Material and Methods

Cloning and Expression of the Candida Hsp90 protein

To clone and express the Candida Hsp90 protein, the coding sequence was PCR amplified directly from Candida genomic DNA, prepared using DNeasy™ spin columns (Qiagen) according to the manufacturer's instructions. Oligonucleotides used were 5′-ATGGCTGACGCAAAAGTTG-3′ (SEQ ID NO: 8) and 5′-ATCAACTTCTTCCATAGCAG -3′ (SEQ ID NO: 9) synthesized by Sigma genosys. Amplification was carried out using Taq DNA polymerase (Invitrogen) allowing direct ligation-independent cloning in to the expression vector pYES2.1/V5-His-TOPO® (Invitrogen), adding a C-terminally fused His₆-tag to the expressed Hsp90 protein under the control of the GAL1 promoter. The cloning mix was transformed in to the E.coli expression strain TOP10F′ (Invitrogen) and recombinants identified using SDS-PAGE and immunoblotting using a monoclonal anti-His-tag peroxidase-conjugate antibody (Sigma). The resulting plasmid was called pHsp1

Purification of the Candida Hsp90 Protein

For over expression of the 6-His-tag Hsp90 protein, Saccharomyces cerevisiae strain INVSc1 was transformed with pHsp1 using the S. c. EasyComp™ kit (Invitrogen) according to the manufacturers instructions. INVSc1 (pHsp1) was grown overnight in 10 ml of SC-U growth medium (0.67% yeast nitrogen base (SIGMA cat. Y-0626), 0.19% yeast synthetic drop-out medium supplement, without uracil (SIGMA cat.Y-1501), 2% Raffinose). Cells were harvested by centrifugation (5000 g, 10 min 4° C.) and the pellet was washed in 10 ml of Sc-U induction medium (0.67% yeast nitrogen base (SIGMA cat. Y-0626), 0.19% yeast synthetic drop-out medium supplement, without uracil (SIGMA cat.Y-1501), 2% Galactose). The washed cells were resuspended in 10 ml of SC-U induction medium and added to 1 L of SC-U induction medium and grown with shaking at 30° C. for a further 24 hours. Cells were harvested by centrifugation (10000 g, 10 min 4° C.) and resuspended in 20 ml of breaking buffer (50 mM sodium phosphate, pH7.4, 5% glycerol, 1 mM PMSF) and broken by French Pressing (2 ton, 1 passage). Insoluble material was removed by a further centrifugation step (10000 g, 10 min at room temperature). The cell Lysate was buffer adjusted with the addition of 500 mM urea and the pH adjusted to pH8.0.

The Hsp90 protein was purified using immobilized metal ion affinity chromatography (IMAC). A 15 ml pre-charged Nickel IMAC column was equilibrated with 5 column volumes (CV) of equilibration buffer (500 mM urea, 100 mM NaH₂PO₄, pH8.0). The buffer adjusted cell Lysate was then applied to the column. The column was washed with 5 CV of equilibration buffer followed by 5 CV of wash buffer (500 mM urea, 100 mM NaH₂PO₄, pH8.0, 50 mM Imidazole) The Hsp90 protein was eluted from the column with 3 CV of elution buffer (500 mM urea, 100 mM NaH₂PO₄, pH8.0, 500 mM Imidazole). All fractions were analyzed by SDS-PAGE, the gel is shown below.

Antibody Sources

Mycograb® is a human recombinant antibody fragment against hsp 90. The epitope to which it binds is conserved between human and fungal hsp90. Aurograb® is a human recombinant antibody against the ABC transporter protein from MRSA. The formulation of Aurograb® is disclosed in WO-A-03/046007, which is hereby incorporated by reference.

Experimental Protocolfor Injection

Female CD-1 mice were used aged 6-8 weeks, usually weighing between 24 and 30 g. Mice were weighed 24 hours prior to each experiment. Concentrations of hsp90 and Mycograb® and Aurograb® were calculated based on mouse weights, at 0.1, 0.5, 1.0 and 10 mg/kg. Control samples were sterile PBS (for hsp90) and sterile formulation buffer (500 mM Urea, 200 mM Arginine pH 9.5) (for Mycograb®). When used in combination, hsp90 and Mycograb® were cross-absorbed at 37° C. for 15 minutes prior to injection.

All mice were placed in a thermo heating box at 41° C. Mice were injected intravenously via the lateral tail vein with the appropriate sample and placed back into cages where they were allowed food and water freely. At specified time points, mice were put under terminal anaesthesia (using halothane). Blood was withdrawn using a sterile needle into the heart (cardiac puncture) and the mouse culled by cervical dislocation.

Blood samples were spun at 3000 rpm for 10 minutes and serum aspirated using a sterile pipette. Serum samples were stored at −20° C. until required for testing.

TNF-α Assay

These were performed according to BD OptEIA™ Catalogue Number 555268 for the mouse values (BD Biosciences Pharmingen San Diego USA). In each case the reaction was performed as per the Manufacturers instructions. A standard curve was required in each assay run. All samples and standards were run in duplicate.

An ELISA plate was coated with 100 μl/well of capture antibody diluted in coating buffer (for recommended dilution see lot-specific certificate of analysis). The plate was sealed and incubated overnight at 4° C. The wells were aspirated and washed three times with wash buffer. After the last wash the plate was inverted and blotted on absorbent paper. The plates were blocked with 200 μl/well of assay diluents for 1 hour at room temperature. The plates were washed three times as previous. The TNF-α standards were prepared as below:

After warming to room temperature the lyophilized standards were reconstituted with 1 ml of deionised water and allowed to equilibrate for 15 minutes before being vortexed to mix. A 1000 pg/ml standard from the stock standard was prepared (dilution instructions are on lot-specific certificate of analysis). From this stock doubling dilutions from 1000 pg/ml to 15.6 pg/ml was prepared using assay diluents. Assay diluent was used as a negative control.

100 μl of each standard, sample and control was added to appropriate wells. The plate was sealed and incubated for two hours at room temperature. Due to the low volumes of sera available the mouse sera was diluted ½ in assay diluents. The plated was washed as previous but with a total of five washes. The required volume of detection antibody was added to assay diluent and vortexed to mix. Just before use the required volume of enzyme reagent was added to the solution and vortexed to mix.

100 μl of working detection antibody was added to each well. The plate was sealed and incubated for one hour at room temperature. The plate was washed as previous but with a total of seven washes.

Substrate was prepared by adding equal volumes of substrate A and substrate B immediately before 100 μl was added to each well. The plate was incubated in the dark for 30 minutes. The reaction was stopped by adding 50 μl of stop solution to each well. The plate was read at 450 nm. The TNF-α concentrations for the samples were determined from the standard curve.

Interleukin 6 Assays

These were performed according to BD OptEIA™ Reagent Set B Catalogue Number 550534 for the human sera and BD OptEIA™ Catalogue Number 555240 for the mouse values (BD Biosciences Pharmingen San Diego USA). In each case the reaction was performed as per the Manufacturers instructions. A standard curve was required in each assay run. All samples and standards were run in duplicate.

An ELISA plate was coated with 100 μl/well of capture antibody diluted in coating buffer (for recommended dilution see lot-specific certificate of analysis). The plate was sealed and incubated overnight at 4° C. The wells were aspirated and washed three times with wash buffer. After the last wash the plate was inverted and blotted on absorbent paper. The plates were blocked with 200 μl/well of assay diluents for 1 hour at room temperature. The plates were washed three times as previous. The IL-6 standards were prepared as below:

After warming to room temperature the lyophilized standards were reconstituted with 1 ml of deionised water and allowed to equilibrate for 15 minutes before being vortexed to mix. A 1000 pg/ml standard from the stock standard was prepared (dilution instructions are on lot-specific certificate of analysis). From this stock doubling dilutions from 1000 pg/ml to 15.6 pg/ml was prepared using assay diluents. Assay diluent was used as a negative control.

100 μl of each standard, sample and control was added to appropriate wells. The plate was sealed and incubated for two hours at room temperature. Due to the low volumes of sera available the mouse sera was diluted ½ in assay diluents. The plated was washed as previous but with a total of five washes. The required volume of detection antibody was added to assay diluent and vortexed to mix. Just before use the required volume of enzyme reagent was added to the solution and vortexed to mix.

100 μl of working detection antibody was added to each well. The plate was sealed and incubated for one hour at room temperature. The plate was washed as previous but with a total of seven washes.

Substrate was prepared by adding equal volumes of substrate A and substrate B immediately before 100 μl was added to each well. The plate was incubated in the dark for 30 minutes. The reaction was stopped by adding 50 μl of stop solution to each well. The plate was read at 450 nm. The IL-6 concentrations for the samples were determined from the standard curve.

Experiment 1

Purified hsp90 was injected at 1 mg/kg and at 10 mg/kg into mice and two mice were sacrificed at 0, 15, 30, 60, and 120 and for 10 mg/kg, in addition, at 1440 minutes. TNF-α and Interleukin 6 levels were measured as described above.

Results

The results showing TNF-α levels in pg/ml are summarised in Table 1.

TABLE 1

| | 1 mg/kg | 1 mg/kg | 10 mg/kg | 10 mg/kg |
|---|---|---|---|---|
| | HSP 90 | HSP 90 | HSP 90 | HSP 90 |
| Time (min) | Mouse 1 | Mouse 2 | Mouse 1 | Mouse 2 |
| 0 | 0 | 0 | 0 | 0 |
| 15 | 11 | 80 | 66 | 192 |
| 30 | 319 | 447 | 485 | 1083 |
| 60 | 941 | 562 | >2000 | >2000 |
| 120 | 265 | 21 | ND | 428 |
| 1440 | ND | ND | 0 | 0 |

The levels of TNF-α increased in response to the administration of hsp90 at both low and high concentrations with a peak at 60 minutes. There was a greater response after administration of the higher dose of hsp 90.

The results showing Interleukin 6 levels in pg/ml are summarised in Table 2.

TABLE 2

| | 1 mg/kg | 1 mg/kg | 10 mg/kg | 10 mg/kg |
|---|---|---|---|---|
| | HSP 90 | HSP 90 | HSP 90 | HSP 90 |
| Time (min) | Mouse 1 | Mouse 2 | Mouse 1 | Mouse 2 |
| 0 | 0 | 0 | 0 | 0 |
| 15 | 0 | 15 | 8 | 62 |
| 30 | 556 | 411 | 500 | 556 |
| 60 | 1321 | 1297 | 1760 | >2000 |
| 120 | 731 | 1 | ND | 1793 |
| 1440 | ND | ND | 0 | 0 |

The results demonstrated a response detectable after 30 minutes which reached a peak at 60 minutes and was undetectable at 1440 minutes with the higher dose. There was a greater response after administration of the higher dose of hsp 90.

Experiment 2

Mice were injected intravenously with either:
1. 1 mg/kg Mycograb
2. 1 mg/kg Aurograb
3. 1 mg/kg HSP90
4. Formulation Buffer
5. 1 mg/kg HSP90 cross absorbed with 1 mg/kg Mycograb (15 mins @ 37° C.)

Mice culled at 1 hr and 2 hr. Each time point was tested in duplicate and TNF-α and interleukin 6 measured.

Results

The results showing TNF-α concentration in pg/ml are summarised in Table 3.

TABLE 3

| | 1 hour | 1 hour | 2 hour | 2 hour |
|---|---|---|---|---|
| | Mouse 1 | Mouse 2 | Mouse 1 | Mouse 2 |
| Mycograb | 0.1 | 5 | 16 | 55 |
| Aurograb | 34 | 67 | 46 | 184 |
| HSP90 | 454 | 534 | 7 | 22 |
| Form. | 6 | 36 | 43 | 29 |
| Buffer | | | | |
| Cross | 455 | 166 | 130 | 102 |
| absorbed | | | | |

The levels of TNF-α were raised slightly by the injection of Formulation buffer, Mycograb and Aurograb. The response to HSP90 was marked and peaked at 1 hour. Cros-absorption with Mycograb had only a marginal effect at 1 hour and at 2 hours the the two mice were higher.

The results showing IL-6 concentration in pg/ml are summarised in Table 4.

TABLE 4

| | 1 hour | 1 hour | 2 hour | 2 hour |
|---|---|---|---|---|
| | Mouse 1 | Mouse 2 | Mouse 1 | Mouse 2 |
| Mycograb | 15 | 12 | 23 | 24 |
| Aurograb | 9 | 9 | 2 | 40 |
| HSP90 | 1660 | 2223 | 28 | 28 |
| Form. | 4 | 4 | 7 | 9 |
| Buffer | | | | |
| Cross | 420 | 287 | 25 | 30 |
| absorbed | | | | |

The levels of Interleukin 6 were unaffected by the injection of Formulation buffer, Mycograb and Aurograb. The response to HSP90 was marked and peaked at 1 hour. Cross-absorption with Mycograb reduced the level of interleukin 6 at 1 hour.

Experiment 3

15 CD-1 mice at approximately 25 g injected with variable concentrations of hsp90 (0-1 mg/kg) with or without cross-absorption with Mycograb (0-1 mg/kg) at 37° C. for 15 minutes prior to injection. All mice were culled at 1 hour and IL-6 levels were monitored.

Experiment 4

15 CD-1 mice at approximately 25 g injected with variable concentrations of hsp90 (0-1 mg/kg) with or without cross-absorption with Mycograb (0-1 mg/kg) at 37° C. for 15 minutes prior to injection. All mice were culled at 1 hour and IL-6 levels were monitored

Experiment 5

30 CD-1 mice at approximately 25 g injected with variable concentrations of hsp90 (0-1 mg/kg) with or without cross-absorption with Mycograb (0-1 mg/kg) at 37° C. for 15 minutes prior to injection. All mice were culled at 1 hour and IL-6 levels were monitored.

Results

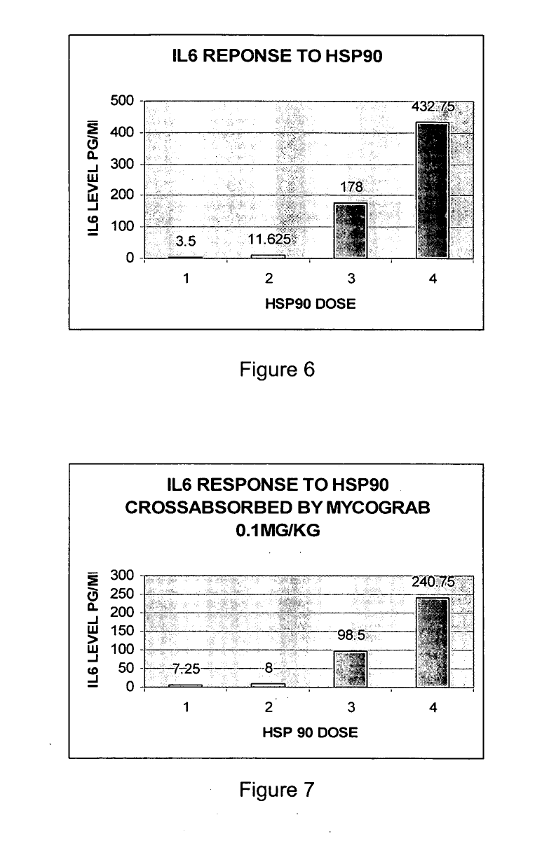

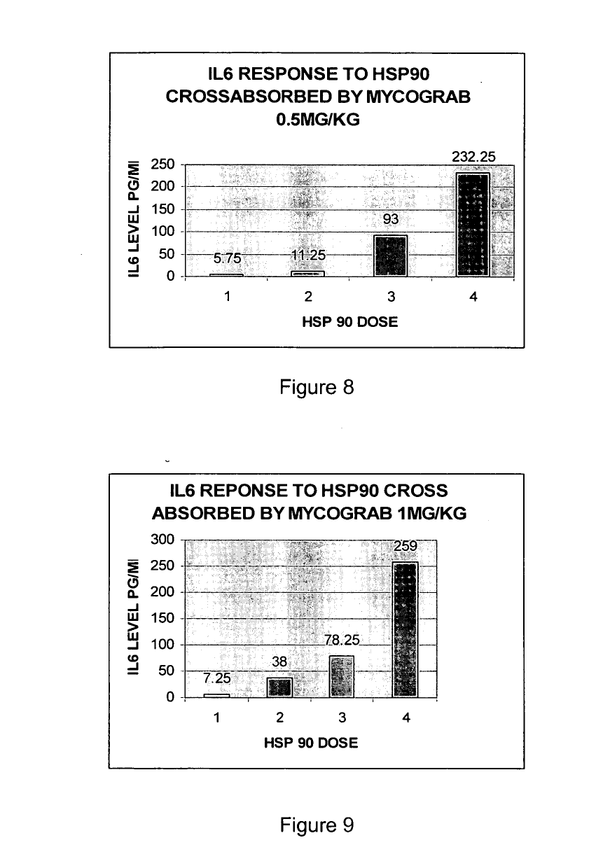

The results from Experiments 3, 4 and 5 are summarised in Table 5 and in FIGS. 6 to 9, in which FIG. 6 shows the IL-6 response to hsp 90; FIG. 7 shows the IL-6 response to hsp 90 when cross-absorbed my Mycograb® at a concentration of 0.1 mg/kg; FIG. 8 shows the IL-6 response to hsp 90 when cross-absorbed my Mycograb® at a concentration of 0.5 mg/kg; and FIG. 9 shows the IL-6 response to hsp 90 when cross-absorbed my Mycograb® at a concentration of 1 mg/kg.

TABLE 5

| | | Experiment 3 | Experiment 4 | Experiment 5 | | IL-6 levels(pg/ml) | |
|---|---|---|---|---|---|---|---|
| HSP 90 | Mycograb | (n = 1) | (n = 1) | (n = 2) | | | Standard |
| (mg/kg) | (mg/kg) | IL-6 (pg/ml) | | | | Mean | deviation |
| 0 | 0 | 3 | 4 | ND | ND | 3.5 | 0.7 |
| 0 | 0.1 | 19 | 3 | 4 | 3 | 7.3 | 7.8 |
| 0 | 0.5 | 8 | 8 | 9 | 6 | 5.8 | 4.0 |
| 0 | 1 | 5 | 12 | 6 | 6 | 7.3 | 3.2 |
| 0.1 | 0 | 12 | 16 | 0.5 | 18 | 11.6 | 7.8 |
| 0.1 | 0.1 | 10 | 10 | 9 | 12 | 8 | 5.4 |
| 0.1 | 0.5 | 34 | 3 | 6 | 2 | 11.3 | 15.3 |
| 0.1 | 1 | 12 | 53 | 37 | 50 | 38 | 18.7 |
| 0.5 | 0 | 88 | 84 | 190 | 350 | 178 | 124.7 |
| 0.5 | 0.1 | 161 | 66 | 133 | 34 | 98.5 | 58.6 |
| 0.5 | 0.5 | 47 | 245 | 41 | 39 | 93 | 101.4 |
| 0.5 | 1 | 103 | 111 | 41 | 58 | 78.3 | 34.1 |
| 1 | 0 | 654 | 657 | 80 | 340 | 432.8 | 278.3 |
| 1 | 0.1 | 359 | 296 | 170 | 138 | 240.8 | 104.2 |
| 1 | 0.5 | 328 | 352 | 123 | 126 | 232.3 | 124.8 |
| 1 | 1 | 205 | 556 | 48 | 227 | 259 | 213.4 |

These results demonstrated that increasing doses 0, 0.1, 0.5 and 1 mg/kg of injected hsp 90 lead to increasing induction of IL-6. This was blocked in part by cross-absorbing the hsp90 with Mycograb at 0.1, 0.5 or 1 mg/kg prior to injection. This effect was most pronounced at the higher doses of hsp90 injection (0.5 and 1 mg/kg) where there was a reduction to 43.8-59.9% of the original signal.

Conclusion from Examples 1 and 2

The above demonstrates that injection of hsp 90 into mice induced a rise in the levels of TNF-α and Interleukin 6. The latter phenomenon was reversed by prior cross-absorption with Mycograb® in a partially dose dependent manner but not by Aurograb®.

### Example 3

### Patient Studies

Two studies were performed. The first was a pilot study which involved the recruitment of 21 patients (termed Pilot study) and the second a Confirmatory study (termed Confirmatory Study) where of the 139 patients enrolled, from Europe and the US, 117 were in the modified intention-to-treat population. Both studies were double-blind, randomised and conducted to determine whether lipid-associated amphotericin B plus Mycograb® was superior to amphotericin B plus placebo in patients with culture-confirmed invasive candidiasis. Patients received a lipid-associated formulation of amphotericin B plus a 5 days course of Mycograb® or placebo. Inclusion criteria included clinical evidence of active infection at trial entry plus growth of Candida from a clinically significant site within 3 days of initiation of study treatment. The primary efficacy variable was overall response (clinical and mycological resolution) to treatment by day 10.

Material and Methods

Enrolment

To be enrolled patients had to be ≧18 years, and had to have one or more positive Candida cultures from a clinically significant site within the previous three days plus at least one of the following signs at study entry: hyperthermia [>38° C.], hypothermia [<36° C.], tachycardia [>110/min], hypotension [mean blood pressure <70 mmHg], high white cell count [>11000/mm³], left shift, need for vasopressor agents or other abnormalities consistent with an ongoing infectious disease process. Significant sites included blood cultures and/or cultures from a deep, normally sterile, site.

Study Procedures

After enrolment, patients were randomly assigned to receive either intravenous Mycograb® (1 mg per kg body weight) or placebo (saline) every 12 hours for 5 days. In addition, each patient was treated with the manufacturer's recommended dose of either Abelcet (5 mg/kg daily) or Ambisome (3 mg/kg daily) for a minimum of 10 days. Patients and investigators remained blinded throughout the study. Apart from systemic antifungal therapy, no other concomitant medications were censored.

Both mycological and clinical responses were used in the assessment of efficacy. Study drug (Mycograb® or placebo) was given for 5 days (days 1-5) and cultures taken on days 2, 3, 4, 5, 6, 8 and 10, or until the signs and symptoms of infection had resolved and cultures were repeatedly negative. Clinical response to treatment was assessed on days 4, 5, 6, 8, 10 and 33 and the course of the disease over the previous 24 hours assessed on a daily basis up until day 10. The assessment of clinical response was made by the local investigator and considered complete if all signs and symptoms thought to be due to the Candida infection had resolved. Hematology, clinical chemistry, coagulation profile and urinalysis were performed at screening and on days 1, 2, 4, 6 and 10.

Evaluation of Efficacy

The primary efficacy endpoint was overall response to treatment on day 10, this being 5 days after the last dose of study drug and the minimum duration of therapy with L-amphotericin. A favourable overall response was defined as a complete clinical and mycological response, with resolution of all signs and symptoms of candidiasis and culture-confirmed eradication. Partial improvement, lack of progress or worsening of the candidiasis were classified as unfavourable.

Patients were thus subdivided into those where the infection resolved (termed “Cured”) and those where it was not (termed “Fail”). Patients who survived at three months were termed “Survivors” and this included some patients who had not made a full response by Day 10.

A further subset was patients who died (termed “All deaths”) which was subdivided into Candida-attributable mortality (termed “Candida deaths”) and those not due to Candida infection (termed “Non Candida deaths”). Candida-attributable mortality was defined as a fatality in which the investigator stated that candidiasis significantly contributed to death, there being clinical evidence of persistent candidiasis, autopsy evidence, and/or death within 48 hours of a positive blood culture (Pappas et al 2003).

Interleukin 6 levels

These were measured as described above. Serum was available from a variable number of patients at entry to the study (Day 1) at the midpoint (Day 3) and on the last day of Mycograb or saline therapy (Day 6). These were analysed according to whether they came from the Pilot study or Confirmatory study and then the two sets of data were combined to produce a Meta analysis (Confirmatory/Pilot).

Statistical Analysis

Mean Values

The mean values from the different patient groups were compared by Mann-Whitney Test with a cutoff of P<0.05 (Graph Pad InStat version 3.0). The mean results from Day 1 were compared to Days 3 and 6 and the results from Day 3 compared to Day 6.

Predictive Analysis

In the case of the patients who died the ability of a high level of interleukin 6 to predict death from Candida attributable or non Candida mortality was examined by Receptor Operating Characteristic Curves (Bewick et al 2004). This compared the levels in patients who died with survivors to answer the question of whether an initial high level of interleukin 6 on day 1 would predict subsequent death and if this differed between patients dieing from Candida versus non-Candida mortality. In the Placebo group this should be predictive as a high interleukin 6 due to circulating hsp 90 would persist. In the Mycograb® group this hsp 90 would be neutralised by Mycograb® and thus the level of initial interleukin 6 would no longer be predictive. This was examined in the Placebo group both for overall mortality and after splitting the patients into Candida and Non-Candida attributable mortality. In the Mycograb® group there were too few patients for this sub-analysis.

The mean levels on Day 1 for the Confirmatory/Pilot patients who died on Mycograb® was 235±327 pg/ml which was similar to the Placebo group 225±307 pg/ml (Tables 8 and 11).

Results

Comparison of Means

These have been summarised in the Tables. Tables 6-8 summarise the results in the Mycograb® group.

The results shown in Table 6 demonstrated a reduction which was statistically significant for the Pilot group in all patients and in the Cured group when the results from Day 1 were compared to those from Days 3 and 6.

The results shown in Table 7 demonstrated a reduction which was statistically significant for the Confirmatory group in all patients and in the Survivor group when the results from Day 1 were compared to those from Day 6.

The results shown in Table 8 demonstrated a reduction which was statistically significant for the Confirmatory/Pilot group in all patients, patients Cured at Day 10 and in the Survivor group when the results from Day 1 were compared to those froms Day 3 and Day 6.

Tables 9 to 11 showed no statistically significant change in the levels in the Placebo group.

TABLE 6
Results of the Pilot study for the Mycograb ® group

| | Pilot Study | | | | | | | | | | | |
|---|---|---|---|---|---|---|---|---|---|---|---|---|
| | | | | | | | | | | Day 1 v | Day 1 v | Day 3 v |
| Mycograb | Mean | SD | No | Mean | SD | No | Mean | SD | No | Day 3 | Day 6 | Day 6 |
| group | Day 1 | | | Day 3 | | | Day 6 | | | P value | P value | P value |
| Pilot | 460 | 529 | 8 | 44 | 30 | 6 | 67 | 35 | 8 | 0.008 | 0.0209 | NS |
| Pilot | 684 | 568 | 5 | 40 | 28 | 3 | 65 | 40 | 5 | 0.0357 | 0.0079 | NS |
| Cured d 10 | | | | | | | | | | | | |
| Pilot | 87 | 55 | 3 | 48 | 38 | 3 | 70 | 34 | 3 | NA | NA | NA |
| Failed d 10 | | | | | | | | | | | | |
| Pilot All | 255 | 205 | 2 | 47 | 20 | 2 | 37 | 14 | 2 | NA | NA | NA |
| deaths (all | | | | | | | | | | | | |
| noncan) | | | | | | | | | | | | |

TABLE 7
Results of the Confirmatory study for the Mycograb ® group

| | Confirmatory Study | | | | | | | | | | | |
|---|---|---|---|---|---|---|---|---|---|---|---|---|
| | | | | | | | | | | Day 1 v | Day 1 v | Day 3 v |
| Mycograb | Mean | SD | No | Mean | SD | No | Mean | SD | No | Day 3 | Day 6 | Day 6 |
| group | Day 1 | | | Day 3 | | | Day 6 | | | P value | P value | P value |
| Confirmatory | 212 | 318 | 52 | 134 | 224 | 47 | 100 | 140 | 50 | NS | 0.0208 | NS |
| Confirmatory | 187 | 302 | 43 | 120 | 219 | 41 | 98 | 139 | 42 | NS | NS | NS |
| Cured d 10 | | | | | | | | | | | | |
| Confirmatory | 331 | 328 | 9 | 229 | 254 | 6 | 114 | 159 | 8 | NS | NS | NS |
| Failed d 10 | | | | | | | | | | | | |
| Confirmatory | 233 | 338 | 24 | 164 | 307 | 22 | 134 | 191 | 22 | NS | NS | NS |
| All deaths | | | | | | | | | | | | |
| Confirmatory | 232 | 354 | 22 | 170 | 313 | 21 | 137 | 195 | 21 | NS | NS | NS |
| Non Candida | | | | | | | | | | | | |
| deaths | | | | | | | | | | | | |
| Confirmatory | 243 | 10 | 2 | 37 | 0 | 1 | 54 | 0 | 1 | NA | NA | NA |
| Candida deaths | | | | | | | | | | | | |
| Survivors | 194 | 286 | 28 | 107 | 109 | 25 | 74 | 75 | 28 | NS | 0.0306 | NS |
| Confirmatory | | | | | | | | | | | | |

TABLE 8
Results of the Confirmatory/Pilot study for the Mycograb ® group

| | Confirmatory and Pilot Study | | | | | | | | | | | |
|---|---|---|---|---|---|---|---|---|---|---|---|---|
| | | | | | | | | | | Day 1 v | Day 1 v | Day 3 v |
| | Mean | SD | No | Mean | SD | No | Mean | SD | No | Day 3 | Day 6 | Day 6 |
| Mycograb group | Day 1 | | | Day 3 | | | Day 6 | | | P value | P value | P value |
| Confirmatory/Pilot | 245 | 350 | 60 | 124 | 213 | 53 | 96 | 131 | 58 | 0.0064 | 0.0028 | NS |
| Confirmatory/Pilot | 239 | 364 | 48 | 114 | 212 | 44 | 94 | 131 | 47 | 0.0335 | 0.04 | NS |
| Cured d 10 | | | | | | | | | | | | |
| Confirmatory/Pilot | 270 | 301 | 12 | 169 | 221 | 9 | 102 | 136 | 11 | NS | NS | NS |
| Failed d 10 | | | | | | | | | | | | |
| Confirmatory/Pilot | 235 | 327 | 26 | 154 | 295 | 24 | 126 | 184 | 24 | NS | NS | NS |
| All deaths | | | | | | | | | | | | |
| Confirmatory/Pilot | 234 | 341 | 24 | 159 | 301 | 23 | 129 | 188 | 23 | NS | NS | NS |
| Non Candida deaths | | | | | | | | | | | | |
| Confirmatory | 243 | 10 | 2 | 37 | 0 | 1 | 54 | 0 | 1 | NA | NA | NA |
| Candida deaths | | | | | | | | | | | | |
| (pilot included- | | | | | | | | | | | | |
| no can death) | | | | | | | | | | | | |
| Survivors | 253 | 372 | 34 | 98 | 104 | 29 | 74 | 70 | 34 | 0.0361 | 0.0075 | NS |
| Confirmatory/Pilot | | | | | | | | | | | | |

TABLE 9
Results of the Pilot study for the Placebo group

| | Pilot Study | | | | | | | | | | | |
|---|---|---|---|---|---|---|---|---|---|---|---|---|
| | | | | | | | | | | Day 1 v | Day 1 v | Day 3 v |
| Placebo | Mean | SD | No | Mean | SD | No | Mean | SD | No | Day 3 | Day 6 | Day 6 |
| group | Day 1 | | | Day 3 | | | Day 6 | | | P value | P value | P value |
| Pilot | 337 | 174 | 8 | 337 | 513 | 8 | 174 | 151 | 8 | NS | NS | NS |
| Pilot | 317 | 247 | 3 | 55 | 40 | 3 | 122 | 72 | 3 | NS | NS | NS |
| Cured d 10 | | | | | | | | | | | | |
| Pilot | 349 | 148 | 5 | 506 | 604 | 5 | 205 | 185 | 5 | NS | NS | NS |
| Failed d 10 | | | | | | | | | | | | |
| Pilot All | 395 | 121 | 4 | 608 | 646 | 4 | 229 | 205 | 4 | NS | NS | NS |
| deaths | | | | | | | | | | | | |
| Pilot Non | 221 | 0 | 1 | 46 | 0 | 1 | 91 | 0 | 1 | NA | NA | NA |
| Can death | | | | | | | | | | | | |
| Pilot Can | 454 | 44 | 3 | 795 | 644 | 3 | 275 | 224 | 3 | NS | NS | NS |
| deaths | | | | | | | | | | | | |

TABLE 10
Results of the Pilot study for the Confirmatory group

| | Confirmatory Study | | | | | | | | | | | |
|---|---|---|---|---|---|---|---|---|---|---|---|---|
| | | | | | | | | | | Day 1 v | Day 1 v | Day 3 v |
| Placebo | Mean | SD | No | Mean | SD | No | Mean | SD | No | Day 3 | Day 6 | Day 6 |
| group | Day 1 | | | Day 3 | | | Day 6 | | | P value | P value | P value |
| Confirmatory | 167 | 245 | 57 | 178 | 342 | 48 | 165 | 292 | 54 | NS | NS | NS |
| Confirmatory | 102 | 109 | 29 | 142 | 343 | 26 | 107 | 230 | 28 | NS | NS | NS |
| Cured d 10 | | | | | | | | | | | | |
| Confirmatory | 234 | 321 | 28 | 221 | 343 | 22 | 228 | 341 | 26 | NS | NS | NS |
| Failed d 10 | | | | | | | | | | | | |
| Confirmatory | 225 | 307 | 21 | 282 | 387 | 16 | 319 | 432 | 20 | NS | NS | NS |
| All deaths | | | | | | | | | | | | |
| Confirmatory | 111 | 121 | 12 | 189 | 225 | 10 | 237 | 359 | 12 | NS | NS | NS |
| Non Candida | | | | | | | | | | | | |
| deaths | | | | | | | | | | | | |
| Confirmatory | 378 | 411 | 9 | 438 | 558 | 6 | 443 | 524 | 8 | NS | NS | NS |
| Candida deaths | | | | | | | | | | | | |
| Survivors | 133 | 198 | 36 | 126 | 310 | 32 | 74 | 84 | 34 | NS | NS | NS |
| Confirmatory | | | | | | | | | | | | |

TABLE 11
Results of the Confirmatory/Pilot study for the Placebo group

| | Confirmatory and Pilot Study | | | | | | | | | | | |
|---|---|---|---|---|---|---|---|---|---|---|---|---|
| | | | | | | | | | | Day 1 v | Day 1 v | Day 3 v |
| | Mean | SD | No | Mean | SD | No | Mean | SD | No | Day 3 | Day 6 | Day 6 |
| Placebo group | Day 1 | | | Day 3 | | | Day 6 | | | P value | P value | P value |
| Confirmatory/Pilot | 188 | 243 | 65 | 200 | 369 | 56 | 166 | 277 | 62 | NS | NS | NS |
| Confirmatory/Pilot | 122 | 137 | 32 | 133 | 326 | 29 | 108 | 219 | 31 | NS | NS | NS |
| Cured d 10 | | | | | | | | | | | | |
| Confirmatory/Pilot | 251 | 303 | 33 | 273 | 405 | 27 | 224 | 318 | 31 | NS | NS | NS |
| Failed d 10 | | | | | | | | | | | | |
| Confirmatory/Pilot | 252 | 290 | 25 | 347 | 449 | 20 | 304 | 401 | 24 | NS | NS | NS |
| All deaths | | | | | | | | | | | | |
| Confirmatory/Pilot | 397 | 353 | 12 | 557 | 575 | 9 | 397 | 456 | 11 | NS | NS | NS |
| Candida deaths | | | | | | | | | | | | |
| Confirmatory/Pilot | 119 | 120 | 13 | 176 | 218 | 11 | 226 | 346 | 13 | NS | NS | NS |
| Non Candida deaths | | | | | | | | | | | | |
| Survivors | 147 | 202 | 40 | 119 | 293 | 36 | 79 | 83 | 38 | NS | NS | NS |
| Confirmatory/Pilot | | | | | | | | | | | | |

Predictive Statistics

The mean levels on Day 1 for the Confirmatory/Pilot patients who died on Mycograb® was 235±327 pg/ml which was similar to the Placebo group 225±307 pg/ml (Tables 8 and 11). The mean values for survivors 253±372 pg/ml for Mycograb® was slightly higher than the 147±202 pg/ml for the Placebo group.

Comparison of the results was based on the AUROC (the area under the curve) (see Table 12), generated by a plot of sensitivity versus 1-Specificity using Graph Pad Prism 4 Soft ware.

TABLE 12
Receiver operator characteristic curves for Interleukin 6

| | | | | 95% Confidence | |
|---|---|---|---|---|---|
| | | | | interval | |
| Comparator | | Standard | | Lower | Upper |
| Groups | AUROC | error | P | bound | bound |
| Mycograb: All | 0.5202 | 0.08060 | 0.7945 | 0.3622 | 0.6782 |
| Deaths versus | | | | | |
| Survivors | | | | | |
| Placebo: All | 0.5960 | 0.07610 | 0.1956 | 0.4468 | 0.7452 |
| Deaths versus | | | | | |
| Survivors | | | | | |
| Placebo: | 0.7552 | 0.07937 | 0.007827 | 0.5996 | 0.9108 |
| Candida Deaths | | | | | |
| versus Survivors | | | | | |
| Placebo: Non | 0.5510 | 0.1002 | 0.5838 | 0.3544 | 0.74775 |
| Candida Deaths | | | | | |
| versus Survivors | | | | | |

Conclusion

The ideal test would have an AUROC of 1, whereas a random guess would have an AUROC of 0.5. This data demonstrated for the Mycograb® group a low predictive value (0.5202). This was consistent with the neutralisation of hsp 90 by Mycograb® meaning that the effect of a high interleukin 6 in altering outcome had been negated. A similar figure (0.5510) was seen when the non Candida deaths in the Placebo group were compared to survivors. This picture changed in the Candida attributable deaths where the AUROC value was 0.7552. This demonstrated that a high interleukin 6 in the absence of Mycograb® to neutralize the circulating hsp 90 led to a much higher chance of death due to Candida.

### REFERENCES

Bewick, V. et al. Critical Care December 2004 Vol 8 No 6, 508-512
Hehlgans, T. et al. Immunology, 115, 1-20
Matthews, R. C. et al. Current Molecular Medicine 2005, 5, 403-411
Miyaoka, K. et al. Journal of Surgical Research 125, 144-150 (2005)
Mokart, D. et al. British Journal of Anaesthesia 94 (6): 767-73 (2005)
Ng, P. C. et al. Arch. Dis. Child. Fetal Neonatal Ed. 1997;77;221-227
Ng, P. C. et al. Arch. Dis. Child. Fetal Neonatal Ed. 2003; 88; 209-213
Ng, P. C. et al. Arch. Dis. Child. Fetal Neonatal Ed. 2004; 89; 229-235
Panacek, E. A. et al. Crit Care Med 2004 Vol. 32, No. 11; 2173-2182
Saito, K. et al. Experimental Cell Research 2005
Terregino, C. A. et al. Annals of Emergency Medicine, 35: 1, Jan. 2000; 26-34

What is claimed is:

1. A method of lowering TNFα or IL-6 levels in a patient comprising administering to the patient an inhibitor of an hsp 90 protein in an amount sufficient to lower the patient's levels of TNFα or IL-6.

2. A method according to claim 1, wherein the patient is suffering from a condition due to raised TNFα or IL-6 levels.

3. A method according to claim 1, wherein the inhibitor comprises an antibody or an antigen-binding fragment thereof.

4. A method of diagnosing a condition in a patient involving raised levels of TNFα or IL-6 comprising the step of determining the level of an hsp 90 protein circulating in the patient, wherein a raised level of the hsp 90 protein is indicative of the presence of the condition.

5. A method according to claim 4, wherein the step of determining the level of the hsp 90 protein circulating in the patient comprises determining the level of the hsp 90 protein in a sample obtained from the patient.

6. A method according to claim 4, wherein the step of determining the level of the hsp 90 protein circulating in the patient comprises binding an antibody or an antigen-binding fragment thereof to the hsp 90 protein.

7. A method according to claim 2 or 4, wherein the condition comprises sepsis, SIRS or an autoimmune disease.

8. A method according to claim 2 or 4, wherein said condition comprises Crohn's disease, rheumatoid arthritis, ulcerative colitis or systemic lupus erythematosus.

9. A method according to claim 7, wherein the sepsis is sepsis due to an infection.

10. A method according to claim 9, wherein the infection is a bacterial or fungal infection.

11. A method according to claim 7, wherein the sepsis is not due to a fungal infection.

12. A method according to claim 7, wherein the sepsis is not due to a bacterial infection.

13. A method according to claim 7, wherein the sepsis is not due to infection.

14. A method according to claim 1 or 4, wherein the hsp 90 protein comprises the amino acid sequence XXXLXVIRKXIV, wherein X is any amino acid (SEQ ID NO: 6).

15. A method according to claim 1 or 4, wherein the hsp 90 protein comprises the amino acid sequence XXILXVIXXXXX, wherein X is any amino acid (SEQ ID NO: 7).

16. A method according to claim 1 or 4, wherein the hsp 90 protein comprises the amino acid sequence LKVIRK (SEQ ID NO: 4).

17. A method according to claim 1 or 4, wherein the hsp 90 protein has at least 50%, 60%, 70%, 80%, 90% or 95% identity to SEQ ID NO: 2.

18. A method according to claim 3 or 6, wherein the antibody or antigen-binding fragment is capable of binding or being specific for an epitope having the amino acid sequence LKVIRK (SEQ ID NO: 4).

19. A method according to claim 18, wherein the antibody comprises the sequence of SEQ ID NO: 1.
